SEQUENCE LISTING (11 sequences)

SEQ ID NO: 1 - DNA, 20 bp, Artificial Sequence
  IL-13 (forward)
atgcccaacaaagcagagac

SEQ ID NO: 2 - DNA, 20 bp, Artificial Sequence
  IL-13 (reverse)
tgagagaaccagggagctgt

SEQ ID NO: 3 - DNA, 24 bp, Artificial Sequence
  TNF-a (forward)
ggctttccgaattcactggagcct

SEQ ID NO: 4 - DNA, 29 bp, Artificial Sequence
  TNF-a (reverse)
ccccggccttccaaataaatacattcata

SEQ ID NO: 5 - DNA, 20 bp, Artificial Sequence
  IL-17 (forward)
tctcatccagcaagagatcc

SEQ ID NO: 6 - DNA, 20 bp, Artificial Sequence
  IL-17 (reverse)
agtttgggacccctttacac

SEQ ID NO: 7 - DNA, 20 bp, Artificial Sequence
  TARC(forward)
cccatgaagaccttcacctc

SEQ ID NO: 8 - DNA, 20 bp, Artificial Sequence
  TARC(reverse)
actctcggcctacattggtg

SEQ ID NO: 9 - DNA, 24 bp, Artificial Sequence
  MUC5AC(forward)
agaatatctttcaggacccctgct

SEQ ID NO: 10 - DNA, 23 bp, Artificial Sequence
  MUC5AC(reverse)
acaccagtgctgagcatactttt

SEQ ID NO: 11 - DNA, 26 bp, Artificial Sequence
  GAPDH(probe)
catcctgcaccaccaactgcttagcc

(19) United States
(12) Patent Application Publication
(10) Pub. No.: US 2021/0138012 A1
(43) Pub. Date: May 13, 2021

(54) COMPOSITION FOR PREVENTING, AMELIORATING, OR TREATING RESPIRATORY DISEASE COMPRISING SIRAITIA GROSVENORII EXTRACT AS EFFECTIVE COMPONENT

(71) Applicant: KOREA INSTITUTE OF ORIENTAL MEDICINE, Daejeon (KR)

(72) Inventors: Dong Seon KIM, Daejeon (KR); Yoon-Young SUNG, Daejeon (KR); Yun Mi LEE, Daejeon (KR); Eun Jung SON, Sejong-si (KR); Heung Joo YUK, Daejeon (KR); Young-Sil LEE, Daejeon (KR)

(21) Appl. No.: 17/150,168

(22) Filed: Jan. 15, 2021

Related U.S. Application Data
(63) Continuation-in-part of application No. PCT/KR2019/009541, filed on Jul. 31, 2019.

(30) Foreign Application Priority Data
Jul. 31, 2018 (KR) 10-2018-0089194

(51) Int. Cl. A61K 36/42; A61P 11/06; A23L 33/00; A23L 33/105

(52) U.S. Cl. CPC A61K 36/42; A61P 11/06; A23V 2002/00; A23L 33/105; A61K 2236/333; A23L 33/40

(57) ABSTRACT

A method for or treating respiratory disease according to an embodiment includes administering to a subject in need thereof an extract of Siraitia grosvenorii residuals as an effective component. The extract of Siraitia grosvenorii residuals has, in an animal model having asthma induced by ovalbumin (OVA), an effect of reducing airway hyper-responsiveness, inhibiting inflammatory cells in bronchoalveolar lavage fluid (BALF), reducing Th2 cytokines (IL-4, IL-5, and IL-13) in BALF, reducing inflammation and tissue damage in lung tissues, inhibiting contraction of airway smooth muscle and airway inflammation, an effect of inhibiting expression of IL-13, TARC, TNF-α, IL-17, and MUC5AC, which is a gene encoding mucus protein, in lung tissues, an effect of reducing the level of ovalbumin-specific IgE in blood serum, and an effect of reducing ROS in alveolar cells which is generated in excess amount due to fine dust.

### CROSS REFERENCE TO RELATED APPLICATIONS

The present application is a continuation in part application to International Application No. PCT/KR2019/009541 with an International Filing Date of Jul. 31, 2019, which claims the benefit of Korean Patent Application No. 10-2018-0089194, filed in the Korean Intellectual Property Office on Jul. 31, 2018, the entire contents of which are incorporated herein by reference.

### BACKGROUND

### 1. Technical Field

The present invention relates to a composition for preventing, ameliorating, or treating respiratory disease comprising an extract of Siraitia grosvenorii residuals as an effective component.

### 2. Background Art

Asthma, chronic obstructive pulmonary disease, allergic rhinitis, cough and sputum, acute or chronic bronchitis, bronchiolitis, pharyngolaryngitis, tonsillitis, laryngitis, and the like are the representative examples of respiratory disease. Asthma indicates chronic inflammation occurring in respiratory tract, in particular, bronchus. Inflammation caused by asthma can be aggravated by various factors such as air pollution, allergic antigen, cold wind, physical exercise, or respiratory infection. Prolonged inflammation yields deforming and hyper-responsiveness of respiratory tract, showing common symptoms including wheezing (high-pitched or coarse breath sound due to narrowed airway), short breath, cough, and discharge of excess amount of sputum.

Respiratory tract is composed of mucous membrane and bronchial smooth muscle. A large number of secretory glands are present in the mucous membrane to discharge continuously the necessary secretions, and the respiratory tract is narrowed according to contraction of bronchial smooth muscle. Once an inflammatory response is caused by various factors such as air pollution, allergic antigen, cold wind, physical exercise, or respiratory infection, a large amount of secretion is discharged from secretory glands and, as the airway is obstructed by the secretion, the mucous membrane swells toward the inside of the airway, resulting in even narrower airway. Accordingly, severe paroxysmal cough accompanied by wheezing and breathing difficulty occur. In case of paroxysmal response, people may have dry cough and experience a pressure in chest. There are many cases of asthma showing only a symptom of breathing difficulty, chronic cough, and a pressure in chest with unknown reasons without any wheezing, and those symptoms tend to occur suddenly in paroxysmal manner while performing the activities of everyday life.

Meanwhile, Siraitia grosvenorii (i.e., monk fruit) is a perennial plant belonging to Cucurbitaceae which is found in the highlands of Guangdong and Guangxi provinces, China. Fruit of Siraitia grosvenorii has either an egg-shape or a ball-shape with diameter of 4 to 5 cm. In Guilin region of China, Siraitia grosvenorii has been traditionally used as a raw material of fresh drink or a seasoning, and it has been also used in a home remedy to treat sore throat, cough, or troubles occurring in stomach or intestine.

As an exemplary prior art related to Siraitia grosvenorii extract, a pharmaceutical composition for preventing and treating asthma and atopy comprising Siraitia grosvenorii as an effective component is described in Korean Patent Publication No. 2015-0051369. However, so far there is no disclosure of a composition for preventing, ameliorating, or treating respiratory disease comprising an extract of Siraitia grosvenorii residuals as an effective component as it is disclosed in the present invention.

### SUMMARY

The present invention is devised under the circumstances described above. The present invention provides a composition for preventing, ameliorating, or treating respiratory disease comprising an extract of Siraitia grosvenorii residuals as an effective component. Specifically, by finding that the extract of Siraitia grosvenorii residuals has, in an animal model having asthma induced by ovalbumin (OVA), an effect of reducing airway hyper-responsiveness, inhibiting inflammatory cells in bronchoalveolar lavage fluid (BALF), reducing Th2 cytokines (IL-4, IL-5, and IL-13) in BALF, reducing inflammation and tissue damage in lung tissues, inhibiting contraction of airway smooth muscle and airway inflammation, an effect of inhibiting expression of IL-13, TARC, TNF-α, IL-17, and MUC5AC, which is a gene encoding mucus protein, in lung tissues, an effect of reducing the level of ovalbumin-specific IgE in blood serum, and an effect of reducing ROS (reactive oxygen species) in alveolar cells which is generated in excess amount due to fine dust, and also by recognizing that the UPLC profile is different between an extract of Siraitia grosvenorii residuals and Siraitia grosvenorii extract, the present invention is completed.

To achieve one or more the purpose described above, an embodiment of the present invention provides a functional health food composition for preventing or ameliorating respiratory disease comprising an extract of Siraitia grosvenorii residuals as an effective component.

An embodiment of the present invention also provides a pharmaceutical composition for preventing or treating respiratory disease comprising an extract of Siraitia grosvenorii residuals as an effective component.

The present invention relates to a composition for preventing, ameliorating, or treating respiratory disease comprising an extract of Siraitia grosvenorii residuals as an effective component. The extract of Siraitia grosvenorii residuals of the present invention has, in an animal model with asthma induced by OVA, an effect of reducing airway hyper-responsiveness, inhibiting inflammatory cells in BALF, reducing Th2 cytokines (IL-4, IL-5, and IL-13) in BALF, reducing inflammation and tissue damage in lung tissues, inhibiting contraction of airway smooth muscle and airway inflammation, an effect of inhibiting expression of IL-13, TARC, TNF-α, IL-17, and MUC5AC, which is a gene encoding mucus protein, in lung tissues, an effect of reducing the level of ovalbumin-specific IgE in blood serum, and an effect of reducing ROS in alveolar cells which is generated in excess amount due to fine dust. As the inflammation-inhibiting activity of extract of Siraitia grosvenorii residuals of the present invention is better than a Siraitia grosvenorii extract, which is a soluble portion, the extract of the present invention can be used more advantageously.

According to an aspect of the present invention, a method for treating respiratory disease may include administering to a subject in need thereof a composition comprising an extract of Siraitia grosvenorii residuals as an effective component.

According to an aspect of the present invention, the extract of Siraitia grosvenorii residuals may be produced by a process including adding an extraction solvent to dried Siraitia grosvenorii followed by extraction and filtration to remove a supernatant, and adding ethanol to residuals of Siraitia grosvenorii, which remain after removal of the supernatant, to obtain an extract of Siraitia grosvenorii residuals.

According to an aspect of the present invention, the extraction solvent may include at least one of water and C₁-C₄ lower alcohol.

According to an aspect of the present invention, the respiratory disease is selected from the group consisting of asthma, chronic obstructive pulmonary disease, bronchitis, pharyngolaryngitis, tonsillitis, and laryngitis.

According to an aspect of the present invention, the respiratory disease is caused by fine dust.

According to an aspect of the present invention, the composition is prepared in any one formulation selected from a powder, a granule, a pill, a tablet, a capsule, a candy, a syrup, and a drink.

According to an aspect of the present invention, the composition is included in a functional health food.

According to an aspect of the present invention, the composition is a pharmaceutical composition.

According to an aspect of the present invention, the pharmaceutical composition further comprises, in addition to the extract of Siraitia grosvenorii residuals, at least one of a pharmaceutically acceptable carrier, vehicle, and diluent.

### BRIEF DESCRIPTION OF THE DRAWINGS

FIG. 1 shows the result of analyzing the airway hyper-responsiveness according to administration of an extract of Siraitia grosvenorii residuals, in which the analysis was made by using an animal asthma model. In the figure, ** and *** indicate that, compared to the control group, the group administered with an extract of Siraitia grosvenorii residuals of the present invention has lower Penh value with statistical significance (**; p<0.01, and ***; p<0.001).

FIG. 2 shows the result of determining the effect of reducing inflammatory cells in BALF as a result of administering the extract of Siraitia grosvenorii residuals to an animal asthma model. In the figure, ### indicates that, compared to the normal group, number of the inflammatory cells and number of the eosinophils in BALF have increased with statistical significance in the control group treated with ovalbumin (OVA) (p<0.001). * and ** indicate that, compared to the control group, number of the inflammatory cells and number of the eosinophils in BALF have decreased with statistical significance in the positive control group (Mon) and also in the group administered with an extract of Siraitia grosvenorii residuals of the present invention (*; p<0.05, and **; p<0.01).

FIG. 3 shows the result of determining the effect of reducing inflammation in lung tissues as a result of administering the extract of Siraitia grosvenorii residuals to an animal asthma model, in which the determination was made based on H&E staining.

FIG. 4 shows the result of determining the effect of reducing tissue damage, which has been caused by collagen precipitation in lung tissues, as a result of administering the extract of Siraitia grosvenorii residuals to an animal asthma model, in which the determination was made based on MT staining.

FIG. 5 shows the result of determining the effect of reducing airway inflammation as a result of administering the extract of Siraitia grosvenorii residuals to an animal asthma model, in which the determination was made based on H&E staining.

FIG. 6 shows the result of determining the effect of reducing airway obstruction and airway inflammation, which have been caused by contraction of airway smooth muscle, as a result of administering the extract of Siraitia grosvenorii residuals to an animal asthma model, in which the determination was made based on AB/PAB staining.

FIG. 7 shows the result of determining the effect of reducing proinflammatory factors (IL-13, TARC, TNF-α, MUC5AC, IL-17) in lung tissues as a result of administering the extract of Siraitia grosvenorii residuals to animal asthma model. In the figure, ## and ### indicate that, compared to the normal group, the proinflammatory factors (IL-13, TARC, TNF-α, MUC5AC, IL-17) have increased with statistical significance in the control group treated with ovalbumin (OVA) (##; p<0.01, and ###; p<0.001). *, ** and *** indicate that, compared to the control group, the proinflammatory factors (IL-13, TARC, TNF-α, MUC5AC, IL-17) have decreased with statistical significance in the positive control group (Mon) and also in the group administered with an extract of Siraitia grosvenorii residuals of the present invention (*; p<0.05, **; p<0.01, and ***; p<0.001).

FIG. 8 shows the result of determining that the level of ovalbumin-specific IgE in blood serum becomes lower as a result of administering the extract of Siraitia grosvenorii residuals to an animal asthma model. In the figure, ### indicates that, compared to the normal group, the level of ovalbumin-specific IgE has increased with statistical significance in the control group treated with OVA (p<0.001). * and ** indicate that, compared to the control group, the level of ovalbumin-specific IgE has decreased with statistical significance in the positive control group (Mon) and also in the group administered with an extract of Siraitia grosvenorii residuals of the present invention (*; p<0.05, and **; p<0.01).

FIG. 9 shows the result of determining that ROS is reduced as a result of administering an extract of Siraitia grosvenorii residuals of the present invention to MH-S cells in which ROS has been generated in large amount due to fine dust. In the figure, ### indicates that, compared to the normal group, the level of ROS has increased with statistical significance in the control group treated with fine dust (CF) (p<0.001). ** and *** indicate that, compared to the control group, the level of ROS has decreased with statistical significance in the group administered with an extract of Siraitia grosvenorii residuals of the present invention (**; p<0.01, and ***; p<0.001).

FIG. 10 shows UPLC profiles of (A) extract of Siraitia grosvenorii residuals of the present invention, (B) ethanol extract of Siraitia grosvenorii, and (C) water extract of Siraitia grosvenorii.

FIG. 11 shows the result of analyzing the airway hyper-responsiveness according to administration of the extract of Siraitia grosvenorii residuals, in which the analysis was made by using an animal asthma model. In the figure, ** and *** indicate that, compared to the control group, the group administered with an extract of Siraitia grosvenorii residuals of the present invention has lower Penh value with statistical significance (**; p<0.01, and ***; p<0.001). In addition, $ indicates that, compared to the extract of Siraitia grosvenorii, the group administered with an extract of Siraitia grosvenorii residuals of has lower Penh value with statistical significance (p<0.05).

FIG. 12 shows the result of analyzing number of white blood cells according to administration of the extract of Siraitia grosvenorii residuals, in which the analysis was made by using an animal asthma model. In the figure, ### indicates that, compared to the normal group, number of the white blood cells (i.e., WBCs No.) has increased with statistical significance in the control group (OVA-CTL) (p<0.001), and * indicates that, compared to the control group, number of the white blood cells has decreased with statistical significance in the group administered with an extract of Siraitia grosvenorii residuals of the present invention (OVA-50 mg/kg Siraitia grosvenorii residuals) and also in the positive control group (OVA-50 mg/kg ivy leaf) (p<0.05). In addition, $ indicates that, compared to the extract of Siraitia grosvenorii, the group administered with an extract of Siraitia grosvenorii residuals has less number of white blood cells with statistical significance (p<0.05).

### DETAILED DESCRIPTION

The present invention relates to a functional health food composition for preventing or ameliorating respiratory disease comprising an extract of Siraitia grosvenorii residuals as an effective component.

The extract of Siraitia grosvenorii residuals is preferably produced by a method including the followings, but it is not limited thereto:
(1) adding an extraction solvent to dried Siraitia grosvenorii followed by extraction and filtration to remove a supernatant; and
(2) adding ethanol to residuals of Siraitia grosvenorii, which remain after removal of the supernatant, to obtain an extract of Siraitia grosvenorii residuals.

The extraction solvent is preferably water, C₁-C₄ lower alcohol, or a mixture thereof, and more preferably water, but it is not limited thereto. The respiratory disease is preferably a disease which is selected from the group consisting of asthma, chronic obstructive pulmonary disease, bronchitis, pharyngolaryngitis, tonsillitis, and laryngitis, but it is not limited thereto. In addition, the respiratory disease may be a disease caused by fine dust, but it is not limited thereto.

The functional health food composition of the present invention can be produced in any one formulation selected from a powder, a granule, a pill, a tablet, a capsule, a candy, a syrup, and a drink, but it is not limited thereto.

When the functional health food composition of the present invention is used as a food additive, the functional health food composition may be directly added to a food product or used with other food product or food ingredient, and it can be suitably used according to a common method. The effective ingredient can be suitably used based on the purpose of use (i.e., prevention or amelioration). In general, for producing a food product or a drink, the addition amount of the functional health food composition of the present invention is 15 parts by weight or less, and preferably 10 parts by weight or less relative to the raw materials. However, in case of long-term consumption under the purpose of maintaining good health, it can be an amount below the aforementioned range, and, as there is no problem in terms of safety, the effective component may be also used in an amount above the aforementioned range.

Type of the functional health food composition is not particularly limited. Examples of the food to which the functional health food composition can be added include meat, sausage, bread, chocolate, candies, snacks, biscuits, pizza, ramen, other noodles, gums, dairy products including ice cream, various kinds of soup, beverage, tea, drink, alcohol beverage, and vitamin complex, and all functional health food products in general sense are included therein. Furthermore, the functional health food composition of the present invention can be also prepared in the form of food, in particularly functional food. The functional food of the present invention may comprise ingredients that are generally comprised in food, and examples thereof include proteins, carbohydrates, lipids, nutrients, and seasonings. When the functional health food composition of the present invention is prepared in the form of a drink, for example, natural carbohydrates or flavoring agents can be comprised as an additional component other than the effective component. Preferred examples of the natural carbohydrates include monosaccharides (e.g., glucose and fructose), disaccharides (e.g., maltose and sucrose), oligosaccharides, polysaccharides (e.g., dextrin and cyclodextrin), and sugar alcohols (e.g., xylitol, sorbitol, and erythritol). As a flavoring agent, natural flavor (e.g., taumatin and stevia extract) and synthetic flavor (e.g., saccharine and aspartame) can be used. The functional health food composition may further comprise various nutritional supplements, a vitamin, an electrolyte, a flavor, a coloring agent, pectinic acid and a salt thereof, alginic acid and a salt thereof, an organic acid, a protective colloidal thickening agent, a pH adjusting agent, a stabilizer, a preservative, glycerin, alcohol, and a carbonating agent used for carbonated drink. Ratio of those components to be added is, although not particularly important, generally selected within a range of 0.01 to 0.1 part by weight per 100 parts by weight of the functional health food composition of the present invention.

The present invention further relates to a pharmaceutical composition for preventing or treating respiratory disease comprising an extract of Siraitia grosvenorii residuals as an effective component.

The respiratory disease may be a disease that is caused by fine dust, but it is not limited thereto.

The pharmaceutical composition comprising an extract of Siraitia grosvenorii residuals of the present invention is preferably any one formulation that is selected from a capsule, a powder, a granule, a tablet, a suspension, an emulsion, a syrup, and an aerosol, but it is not limited thereto. The pharmaceutical composition of the present invention may further comprise, other than the extract of Siraitia grosvenorii residuals described above, a pharmaceutically acceptable carrier, vehicle, or diluent. Examples of the carrier, vehicle, or diluent which may be comprised in the pharmaceutical composition comprising an extract of Siraitia grosvenorii residuals include lactose, dextrose, sucrose, oligosaccharide, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and mineral oil. In case of preparing a formulation, the preparation can be made by using a diluent or a vehicle like a filling agent, a bulking agent, a binding agent, a wetting agent, a disintegrating agent, and a surfactant that are commonly used. The preferable dosage of the extract of Siraitia grosvenorii residuals of the present invention may be differently set depending on condition and bodyweight of a patient, severeness of disorder, pharmaceutical form, administration pathway and period, and it may suitably set by a person who is skilled in the art. However, to have a desired effect, the pharmaceutical composition of the present invention comprising an extract of Siraitia grosvenorii residuals may be administered in an amount of 0.0001 to 100 mg/kg, and preferably 0.001 to 10 mg/kg per day. The administration can be made once a day, or several times a day with divided portion. The scope of the present invention is not limited by the aforementioned dosage in any sense.

Hereinbelow, the present invention is explained in greater detail in view of the Examples. However, the following Examples are given only for more specific explanation of the present invention and it would be evident to a person who has common knowledge in the pertinent art that the scope of the present invention is not limited by them.

### EXAMPLES

### Example 1. Preparation of Extract of Siraitia grosvenorii

Dried fruit of Siraitia grosvenorii (200 g) was crushed and added with 2 liter of water followed by reflux extraction for 3 hours. To 50 g of dried residuals which have been obtained by filtering the extracted solution and drying the remainings, one liter of 70% (v/v) ethanol was added and the mixture was subjected to reflux extraction for 3 hours. After filtering the extracted solution, an extract of Siraitia grosvenorii residuals was obtained by concentration and drying.

Also, for comparative examples, each dried and crushed Siraitia grosvenorii (200 g) was added with 2 liter of water and 2 liter of 70% ethanol followed by reflux extraction for 3 hours. After filtering the extracted solution, a water extract of Siraitia grosvenorii and a 70% ethanol extract of Siraitia grosvenorii were obtained by concentration and drying.

### Example 2. Analysis of Airway Hyper-Responsiveness According to Administration of Extract of Siraitia grosvenorii Residuals Using Animal Asthma Model

Test was carried out by using an animal model which has been induced to have bronchial asthma by ovalbumin (OVA). As for the test animal, a 7-week old male Balb/c mouse was obtained from Jackson Laboratory (Bar Harbor, Me., USA), and used for the test after acclimation for 1 week. With an interval of 2 weeks, 0.25 ml of phosphate buffer solution in which 0.26 mg of aluminum hydroxide (A8222, Sigma-Aldrich, MO, USA) and 12.5 μg of ovalbumin (A5503, Sigma-Aldrich) are suspended was intraperitoneally administered to the acclimated mouse for sensitization. On day 3 and day 10 after intraperitoneally administering the first OVA, 2 mg of OVA were administered to the mouse via intratracheal injection. Then, from day 21 onwards for 4 weeks, the mouse was allowed to inhale OVA for 30 minutes by using a ultrasonic sprayer (NE-U12, Omron Co., Tokyo, Japan) (week 1 to week 3; exposed to 1% OVA, week 4; exposed to 2% OVA). Forty-eight hours after the last exposure to OVA, blood was taken from the anesthetized mouse, which was then subjected to autopsy to have observation of a pathological change.

The test was carried out by having test groups including a normal group (Normal, group having no administration and no inhalation of OVA), an asthma-induced group (OVA-Control, group having OVA administration and inhalation), positive control group (OVA-Mon, i.e., group having administration of 10 mg/kg montelukast and administration and inhalation of OVA), test sample administration group 1 (OVA-200 mg/kg extract of Siraitia grosvenorii residuals, i.e., group having administration and inhalation of 200 mg/kg extract of Siraitia grosvenorii residuals+OVA), test sample administration group 2 (OVA-100 mg/kg extract of Siraitia grosvenorii residuals, i.e., group having administration and inhalation of 100 mg/kg extract of Siraitia grosvenorii residuals+OVA).

The pharmaceutical and test sample were administered for 4 weeks starting from day 21 after administration of the first OVA. Ten mice were used for each group.

To analyze the airway hyper-responsiveness, 24 hours after exposure to the last OVA, airway hyper-responsiveness caused by occurrence of asthma was measured by using Buxco system (Biosystems XA, DSI, MN, USA). Degree of the airway resistance was evaluated by measuring enhanced pause (Penh). After allowing the animal to inhale methacholine (A2251, Sigma-Aldrich) at gradually increased concentration (i.e., 6.25, 12.5, and 25 mg/ml), Penh value was calculated by using the mathematical formula 1 below.

Penh=PausexPEF/PIF, Pause=(Tₑ−Tᵣ)/Tᵣ Mathematical formula 1:

(PIF: peak inspiratory flow, PEF: peak expiratory flow, Tₑ: e time, Tᵣ: relaxation time).

As the results are shown in FIG. 1, the asthma-induced group has Penh value which increases significantly in proportion to the concentration of methacholine. On the contrary, when the animal was treated with 100 or 200 mg/kg extract of Siraitia grosvenorii residuals of the present invention, lower Penh value than the asthma-induced group was observed. Compared to the treatment of 25 mg/kg methacholine, in particular, a difference with statistical significance was shown.

### Example 3. Determination of Decrease Effect in Inflammatory Cells in BALF (Bronchoalveolar Lavage Fluid) after Administration of Extract of Siraitia grosvenorii Residuals to Animal Asthma Model

Forty-eight hours after the last exposure to OVA, the animal model was anesthetized and the bronchus was cut. Bronchoalveolar washing was carried out with ice-cold DMEM in total amount of 1.0 ml and the resulting lavage fluid was collected. The BALF from each test animal was centrifuged immediately after the collection to separate hemocytes, which were then stained with 0.04% trypan blue. Then, the total cell count was obtained by using a hematocytometer. Sample smearing was carried out using Cytospin followed by Diff-Quick staining (Romanowsky stain). Using an optical microscope (Light microscope, Nikon, Japan, magnification: 400×), eosinophils and other inflammatory cells were separately counted.

As the results are shown in FIG. 2, it was found that the inflammatory cells have increased with statistical significance in BALF of the asthma-induced group. On the contrary, the inflammatory cells have decreased with statistical significance in BALF of the group administered with the extract of Siraitia grosvenorii residuals of the present invention.

### Example 4. Determination of Inhibitory Effect on Production of Th2 Cytokines (IL-4, IL-5, and IL-13) in BALF after Administration of Extract of Siraitia grosvenorii Residuals to Animal Asthma Model

Production amount of interleukins (IL-4, IL-5, and IL-13) in BALF separated from each test animal was measured by a commercially available kit for enzyme-linked immunosorbent assay (ELISA) (R&D System, USA). Analysis of each cytokine was carried out according to the experimental method provided by the manufacturer, and the absorbance at 450 nm was measured using an ELISA reader.

As the results are shown in Table 1, the production amount of IL-4, IL-5, and IL-13 has increased with statistical significance in BALF of the asthma-induced group. On the contrary, it was found that, compared to the asthma-induced group, the production amount of IL-4, IL-5, and IL-13 has decreased with statistical significance in BALF of the group administered with the extract of Siraitia grosvenorii residuals of the present invention.

TABLE 1
Number of inflammatory cells in BALF

| | IL-4 (pg/ml) | IL-5 (pg/ml) | IL-13 (pg/ml) |
|---|---|---|---|
| Normal group (Normal) | 32.39 ± 7.54 | 18.93 ± 8.85 | 29.93 ± 14.19 |
| Asthma-induced group | 137.93 ± 17.53^{###} | 89.22 ± 23.56^{#} | 240.42 ± 25.90^{###} |
| (OVA-Control) | | | |
| Positive control group | 60.37 ± 12.05** | 27.91 ± 8.61* | 89.58 ± 9.46*** |
| (OVA-10 mg/kg Montelukast) | | | |
| OVA-200 mg/kg Siraitia | 86.30 ± 12.05* | 30.55 ± 7.86* | 80.19 ± 30.30** |
| grosvenorii residuals | | | |
| OVA-100 mg/kg Siraitia | 103.37 ± 18.78 | 42.88 ± 8.11 | 105.71 ± 35.30* |
| grosvenorii residuals | | | |

^{#, ###}number of the inflammatory cells has increased with statistical significance in the asthma-induced group compared to the normal group: ^{#}p < 0.05, and ^{###}p < 0.001.
*, **, ***compared to the asthma-induced group, number of the inflammatory cells has decreased with statistical significance in the positive control group and the group treated with extract of Siraitia grosvenorii residuals of the present invention: *p < 0.05, **p < 0.01, and ***p < 0.001

### Example 5. Histopathological Analysis after Administration of Extract of Siraitia grosvenorii Residuals to Animal Asthma Model (H&E, MT, AB/PAS Staining)

To determine the level of pathological damage in lung and airway (trakea) tissues, lung tissues were removed and, according to fixing with 10% neutral buffered formalin and paraffin embedding, a block was prepared, which was then cut to 4 μm thickness to give a tissue specimen. After that, to observe the inflammation in lung and airway tissues, H&E (Hematoxylin & Eosin) staining was carried out, and also MT (Masson's trichrome) staining, which is collagen deposition staining, and AB-PAS (AB-periodic acid Schiff) staining for observing airway obstruction caused by mucus secretion and contraction of airway smooth muscle were carried out. By using an optical microscope, a pathological change in lung and airway was examined.

As the results are shown in FIG. 3 to FIG. 6, it was found that the inflammation and tissue damage in lung tissues, which are exhibited in the asthma-induced group, and also the contraction of airway smooth muscle and airway inflammation have decreased in the group administered with the extract of Siraitia grosvenorii residuals of the present invention.

### Example 6. Determination of Effect of Reducing Proinflammatory Factors (IL-13, TARC, TNF-α, MUC5AC, IL-17) in Lung Tissues after Administration of Extract of Siraitia grosvenorii Residuals to Animal Asthma Model

Lung tissues were collected and total RNA was extracted from the tissues by using RNAzol B reagent (Tel-Test, Austin, Tex., USA). cDNA was then synthesized with 3 μg total RNA by using ReverTraAce-a-cDNA Synthesis kit (Toyobo, Osaka, Japan). Synthesized cDNA was applied to real time polymerase chain reaction (real-time PCR) using Applied Biosystems 7500 Real-time PCR system (Applied Biosystems, USA) to analyze the expression of IL-13, TNF-α, IL-17, TARC, and MUC5AC. Conditions of the real-time PCR include pre-denaturation for 2 minutes at 50° C. and 10 minutes at 94° C., and 40 cycles of 94° C. for 1 minute and 60° C. for 1 minute. As GAPDH probe, CATCCTGCACCACCAACTGCTTAGCC (VIC) (SEQ ID NO: 11) was used (probe was a product supplied by Applied Biosystems). For the test sample administration group and the control group, GAPDH was used as an internal standard and RQ (relative quantitative) was estimated by using the following mathematical formula 2.

Quantitative PCR of target group y=x(1+e)n, Mathematical formula 2

in which x=starting quantity

y=yield

n=number of cycles

e=efficiency.

RQ (relative quantitative) was estimated by using the above.

TABLE 2

| | | Sequence (5′->3′) | |
|---|---|---|---|
| IL-13 | forward | ATGCCCAACAAAGCAGAGAC | SEQ ID NO: 1 |
| | reverse | TGAGAGAACCAGGGAGCTGT | SEQ ID NO: 2 |
| TNF-α | forward | GGCTTTCCGAATTCACTGGA | SEQ ID NO: 3 |
| | | GCCT | |
| | reverse | CCCCGGCCTTCCAAATAAAT | SEQ ID NO: 4 |
| | | ACATTCATA | |
| IL-17 | forward | TCTCATCCAGCAAGAGATCC | SEQ ID NO: 5 |
| | reverse | AGTTTGGGACCCCTTTACAC | SEQ ID NO: 6 |
| TARC | forward | CCCATGAAGACCTTCACCTC | SEQ ID NO: 7 |
| | reverse | ACTCTCGGCCTACATTGGTG | SEQ ID NO: 8 |
| MUC5AC | forward | AGAATATCTTTCAGGACCCC | SEQ ID NO: 9 |
| | | TGCT | |
| | reverse | ACACCAGTGCTGAGCATACT | SEQ ID NO: 10 |
| | | TTT | |
| GAPDH | probe | CATCCTGCACCACCAACTGC | SEQ ID NO: 11 |
| | | TTAGCC | |

As the results are shown in FIG. 7, it was found that expression of the proinflammatory factors (IL-13, TARC, TNF-α, MUC5AC, IL-17) in lung tissues has decreased after administering the extract of Siraitia grosvenorii residuals to an animal asthma model.

### Example 7. Measurement of OVA-Specific Immunoglobulin E in Blood Serum

To measure OVA-specific immunoglobulin E in blood serum, blood collected by cardiac puncture was reacted for 30 minutes at room temperature and subjected to centrifuge (2500 rpm, 15 min) to give blood serum. Measurement of OVA-specific immunoglobulin E in the blood serum was carried out by using ELISA kit (Shibayagi, Japan) according to the manufacturer's protocol. Chromogenic reaction was followed by measuring the absorbance at 450 nm.

As the results are shown in FIG. 8, it was found that level of OVA-specific immunoglobulin E in blood serum has decreased as a result of administering the extract of Siraitia grosvenorii residuals to an animal asthma model.

### Example 8. Determination of Amelioration of Inflammation Caused by Fine Dust

MH-S alveolar macrophage cells were aliquoted in a 96-well plate and cultured. The cells were then treated with fine dust (50 μg/ml) and the extract of Siraitia grosvenorii residuals at different concentrations (25, 50, 100, 200, and 400 μg/ml) followed by culture for 24 hours. The MH-S cells were then separated, added to a FACS tube, washed, and added with DCF-DA solution (10 μM concentration per tube) followed by suspension. After staining for 30 minutes in a dark room at 37° C., ROS was analyzed by using a flow cytometer (FACS Calibur flow cytometry system, BD Biosciences, Mountain View, Calif., USA).

Fine dust is a material which acts on pulmonary epithelial cells and macrophage cells to induce oxidative stress, and causes inflammations by increasing reactive oxygen species (ROS). The fine dust mixture (CF) used in the examples of the present invention was prepared by mixing coal ash, fly ash JIS type-II Fly ash), and diesel exhaust particulate (DEP).

As a result, when alveolar macrophage MH-S cells of mouse were treated with the fine dust mixture, ROS production becomes higher with statistical significance compared to the normal group not treated with any fine dust. However, when treated simultaneously with the extract of Siraitia grosvenorii residuals of the present invention (100 to 400 μg/ml), the increased ROS was reduced with statistical significance (FIG. 9).

### Example 9. Comparison of Component Profile Between Extract of Siraitia grosvenorii Residuals and Extract of Siraitia grosvenorii

To determine that the extract of Siraitia grosvenorii residuals of the present invention is different from an extract of Siraitia grosvenorii, their profiles were compared to each other by using UPLC-quadrupole time of flight mass spectrometry (qTof MS). Specifically, as UPLC, ACQUITY UPLC™ system by Waters (USA) was used, and BEH C₁₈ of ODS series (100×2.1 mm) was used as a column. As a mobile phase, water containing 0.1% formic acid and acetonitrile containing 0.1% formic acid were used, and gradient elution in which acetonitrile solvent increases from 15% at the beginning to 100% over 13 minutes was applied. The flow rate was adjusted to 0.4 ml/min and the amount of injected extraction solution was 2 μl. The detector was qTof MS and analysis was made according to negative ion mode.

As the results are shown in FIG. 10, the profile of the water extract of Siraitia grosvenorii was similar to the profile of the ethanol extract of Siraitia grosvenorii. However, it was found that the ethanol extract of Siraitia grosvenorii residuals of the present invention is different from the water and ethanol extracts of Siraitia grosvenorii. Accordingly, it was recognized that the effective component is different between the two inventions.

### Example 10. Comparison of Secretion Amount of Chemokines (RANTES and TARC) Between Extract of Siraitia grosvenorii Residuals and Extract of Siraitia grosvenorii Using Human Bronchial Epithelial Cells

BEAS-2B cells (ATCC, USA), which are human bronchial epithelial cells, were cultured in Dulbecco's modified eagle medium (DMEM) added with fetal bovine serum (FBS) and penicillin-streptomycin (PS). BEAS-2B cells were cultured again for 18 hours in a 96-well plate (5×10⁴ cells/well) containing DMEM 10% medium. The medium was subsequently removed and replaced with serum-free DMEM.

After that, to measure the secretion amount of RANTES, the cells were simultaneously treated with a test sample and TNF-α (10 ng/ml) and cultured for 24 hours. Secretion amount of RANTES present in cell supernatant was measured by using ELISA kit (R&D systems, USA) according to the manufacturer's protocol. As a positive control group, 0.1 μM dexamethasone was used.

As a result, the rate of inhibiting RANTES secretion by a 70% ethanol extract of Siraitia grosvenorii has almost no difference compared to the control group. However, the rate of inhibiting RANTES secretion by the extract of Siraitia grosvenorii residuals and the inhibition rate by the positive control group are higher with statistical significance compared to the control group (Table 3).

TABLE 3
Comparison of inhibitory effect on secretion of proinflammatory chemokine
RANTES in BEAS-2B pulmonary bronchial cells

| | | | | Statistical |
|---|---|---|---|---|
| | Test sample | | Inhibition | significance (vs. |
| Test sample | concentration | | rate (%) | Control group) |
| Normal group | 0 | | 100.00 ± 6.94 | |
| 10 ng/ml TNF-α treatment (Control | 0 | | 0.00 ± 9.08 | |
| group) | | | | |
| 10 ng/ml TNF-α + Siraitia grosvenorii | 200 | μg/ml | 17.47 ± 10.84 | |
| 10 ng/ml TNF-α + Siraitia grosvenorii | 100 | μg/ml | −2.79 ± 36.62 | |
| 10 ng/ml TNF-α + Siraitia grosvenorii | 50 | μg/ml | 17.96 ± 12.44 | |
| 10 ng/ml TNF-α + Siraitia grosvenorii | 25 | μg/ml | 9.94 ± 6.10 | |
| 10 ng/ml TNF-α + Siraitia grosvenorii | 200 | μg/ml | 102.94 ± 5.74 | ***p < 0.001 |
| residuals | | | | |
| 10 ng/ml TNF-α + Siraitia grosvenorii | 100 | μg/ml | 59.86 ± 4.92 | ***p < 0.001 |
| residuals | | | | |
| 10 ng/ml TNF-α + Siraitia grosvenorii | 50 | μg/ml | 57.66 ± 12.63 | **p < 0.01 |
| residuals | | | | |
| 10 ng/ml TNF-α + Siraitia grosvenorii | 25 | μg/ml | 38.18 ± 6.58 | **p < 0.01 |
| residuals | | | | |
| 10 ng/ml TNF-α + positive control | 0.1 | μM | 97.06 ± 12.92 | ***p < 0.001 |
| group | | | | |

Meanwhile, to measure the secretion amount of TARC, BEAS-2B cells were simultaneously treated with TNF-α (50 ng/ml), IFN-γ (10 ng/ml), and IL-4 (50 ng/ml), and, after culture for 24 hours, the measurement was made by using ELISA kit (R&D systems, USA).

As the results are shown in Table 4, the 70% ethanol extract of Siraitia grosvenorii showed no statistically significant increase in the inhibition rate on TARC secretion when compared to the control group. However, the extract of Siraitia grosvenorii residuals and the positive control group showed a statistically significant increase in the inhibition rate on TARC secretion when compared to the control group.

TABLE 4
Comparison of inhibitory effect on secretion of proinflammatory chemokine
TARC in BEAS-2B pulmonary bronchial cells

| | | | | Statistical |
|---|---|---|---|---|
| | Test sample | | Inhibition | significance (vs. |
| Test sample | concentration | | rate (%) | Control group) |
| Normal group | 0 | | 100.0 ± 1.05 | |
| TNF-α + IFN-γ + IL-4 treatment (Control group) | 0 | | 0.0 ± 18.80 | |
| TNF-α + IFN-γ + IL-4 + Siraitia grosvenorii | 200 | μg/ml | 10.53 ± 12.87 | |
| TNF-α + IFN-γ + IL-4 + Siraitia grosvenorii | 100 | μg/ml | 20.36 ± 9.44 | |
| TNF-α + IFN-γ + IL-4 + Siraitia grosvenorii | 50 | μg/ml | 20.42 ± 12.10 | |
| TNF-α + IFN-γ + IL-4 + Siraitia grosvenorii | 200 | μg/ml | 69.47 ± 12.67 | ***p < 0.001 |
| residuals | | | | |
| TNF-α + IFN-γ + IL-4 + Siraitia grosvenorii | 100 | μg/ml | 43.31 ± 14.87 | *p < 0.05 |
| residuals | | | | |
| TNF-α + IFN-γ + IL-4 + Siraitia grosvenorii | 50 | μg/ml | 41.85 ± 15.09 | *p < 0.05 |
| residuals | | | | |
| TNF-α + IFN-γ + IL-4 + positive control | 0.1 | μM | 28.39 ± 8.77 | *p < 0.05 |
| group | | | | |

Meanwhile, as a result of determining the cytotoxicity of a 70% ethanol extract of Siraitia grosvenorii and an extract of Siraitia grosvenorii residuals to BEAS-2B pulmonary bronchial cells, it was found that there is almost no cytotoxicity as described in Table 5.

TABLE 5
Evaluation of cytotoxicity in BEAS-2B pulmonary
bronchial cells using CCK-8

| | Test sample | | |
|---|---|---|---|
| Test sample | concentration | | Cell viability (%) |
| Normal group | 0 | | 100.00 ± 6.94 |
| Siraitia grosvenorii | 200 | μg/ml | 102.43 ± 0.05 |
| Siraitia grosvenorii | 100 | μg/ml | 101.09 ± 0.03 |
| Siraitia grosvenorii | 50 | μg/ml | 100.44 ± 0.01 |
| Siraitia grosvenorii | 25 | μg/ml | 99.95 ± 0.10 |
| Siraitia grosvenorii residuals | 200 | μg/ml | 107.87 ± 0.10 |
| Siraitia grosvenorii residuals | 100 | μg/ml | 106.16 ± 0.02 |
| Siraitia grosvenorii residuals | 50 | μg/ml | 103.14 ± 0.22 |
| Siraitia grosvenorii residuals | 25 | μg/ml | 98.20 ± 0.17 |
| Positive control group | 0.1 | μM | 99.34 ± 0.14 |

### Example 11. Comparison of Asthma-Inhibiting Activity Between Extract of Siraitia grosvenorii Residuals and Extract of Siraitia grosvenorii Using Animal Asthma Model

The test was carried out in the same manner as the above Example 2. The asthma-inhibiting activity was determined for different groups and compared between the extract of Siraitia grosvenorii residuals and the 70% ethanol extract of Siraitia grosvenorii, in which the test groups include a normal group (Normal, group having no administration and no inhalation of OVA), an asthma-induced group (OVA-Control, group having OVA administration and inhalation), positive control group (OVA-50 mg/kg extract of ivy leaf, i.e., group having administration of 50 mg/kg ivy leaf and administration and inhalation of OVA), test group (OVA-50 mg/kg Siraitia grosvenorii residuals, i.e., 50 mg/kg extract of Siraitia grosvenorii residuals and administration and inhalation of OVA), and comparative group (OVA-50 mg/kg Siraitia grosvenorii, i.e., 50 mg/kg 70% ethanol extract of Siraitia grosvenorii and administration and inhalation of OVA). For 4 weeks from day 21 after the administration of the first OVA, the chemicals and test samples were orally administered. Six mice were employed for each test group.

To evaluate the asthma-inhibiting activity, the airway resistance was determined, and, upon the termination of the test, blood was collected and number of white blood cells (WBC), which is an inflammation indicator, was counted via a complete blood count (CBC) test.

As the results are shown in FIG. 11, the asthma-induced group has Penh value which increases significantly in proportion to the concentration of methacholine. On the contrary, when the animals were treated with 50 mg/kg extract of Siraitia grosvenorii residuals of the present invention, lower Penh value than the asthma-induced group was observed in case of treating with 25 mg/kg methacholine, showing a decrease with statistical significance. It was also found that, compared to the group administered with a 70% ethanol extract of Siraitia grosvenorii, the group treated with an extract of Siraitia grosvenorii residuals has a significantly lower Penh value.

Meanwhile, as a result of determining the number of the white blood cells, which is an inflammation indicator, via a CBC test, it was found that the WBC number has increased significantly in the asthma-induced group as shown in FIG. 12. On the contrary, when the treatment with the extract of Siraitia grosvenorii residuals of the present invention is carried out at concentration of 50 mg/kg, lower WBC number was yielded compared to the asthma-induced group, showing a decrease with statistical significance. It was also found that the group treated with an extract of Siraitia grosvenorii residuals has WBC number that is lower, with statistical significance, than the number from a 70% ethanol extract of Siraitia grosvenorii.

Statistical Processing

All the measurement results are expressed in terms of mean and standard error of the mean (SE), and a difference among the test groups was subjected to a statistical analysis using Student's t-test. Statistical significance was recognized when p is less than 0.05 (p<0.05).

A sequence listing electronically submitted with the present application on Jan. 15, 2021 as an ASCII text file named 20210115_Q47921GR01_TU_SEQ, created on Jan. 12, 2021 and having a size of 3000 bytes, is incorporated herein by reference in its entirety.

What is claimed is:

1. A method for treating respiratory disease, the method comprising administering to a subject in need thereof a composition comprising an extract of Siraitia grosvenorii residuals as an effective component.

2. The method of claim 1, wherein the extract of Siraitia grosvenorii residuals is produced by a process comprising:
adding an extraction solvent to dried Siraitia grosvenorii followed by extraction and filtration to remove a supernatant; and
adding ethanol to residuals of Siraitia grosvenorii, which remain after removal of the supernatant, to obtain an extract of Siraitia grosvenorii residuals.

3. The method of claim 2, wherein the extraction solvent comprises at least one of water and C₁-C₄ lower alcohol.

4. The method of claim 1, wherein the respiratory disease is selected from the group consisting of asthma, chronic obstructive pulmonary disease, bronchitis, pharyngolaryngitis, tonsillitis, and laryngitis.

5. The method of claim 1, wherein the respiratory disease is caused by fine dust.

6. The method of claim 1, wherein the composition is prepared in any one formulation selected from a powder, a granule, a pill, a tablet, a capsule, a candy, a syrup, and a drink.

7. The method of claim 1, wherein the composition is included in a functional health food.

8. The method of claim 1, wherein the composition is a pharmaceutical composition.

9. The method of claim 8, wherein the pharmaceutical composition further comprises, in addition to the extract of Siraitia grosvenorii residuals, at least one of a pharmaceutically acceptable carrier, vehicle, and diluent.

10. The method of claim 8, wherein the respiratory disease is caused by fine dust.
